(19) United States
(12) Patent Application Publication
(10) Pub. No.: US 2026/0207683 A1
(43) Pub. Date: Jul. 23, 2026

(54) GEL PREPARATION, METHOD FOR MAKING THE SAME, AND USE THEREOF

(71) Applicant: Duhui Kangjian(Chengdu)Medical Technology Co.,Ltd., Chengdu (CN)

(72) Inventor: Zhenye ZHANG, Chengdu (CN)

(73) Assignee: Duhui Kangjian(Chengdu)Medical Technology Co.,Ltd., Chengdu (CN)

(21) Appl. No.: 19/029,765

(22) Filed: Jan. 17, 2025

(51) Int. Cl. A61K 35/747; A61K 9/06; A61K 35/644; A61K 36/33; A61K 36/42; A61K 36/489; A61K 36/605; A61K 36/61; A61K 36/752; A61K 36/888; A61K 47/36; A61P 1/04; A61P 3/06; A61P 3/10

(52) U.S. Cl. CPC A61K 35/747; A61K 9/06; A61K 35/644; A61K 36/33; A61K 36/42; A61K 36/489; A61K 36/605; A61K 36/61; A61K 36/752; A61K 36/888; A61K 47/36; A61P 1/04; A61P 3/06; A61P 3/10; A61K 2236/15; A61K 2236/19; A61K 2236/331; A61K 2236/37

(57) ABSTRACT

Provided is a gel preparation, a method for making the same and use thereof. This disclosure belongs to the technical field of functional food. The gel preparation capable of maintaining blood glucose and lipids within a healthy range and assisting in protecting gastric mucosa includes the following raw materials: konjac, a banana, a watermelon, dragon fruit peel, Caryophylli Flos, Mori Folium Praeparata Cum Melle, Sophorae Flos Carbonisatus, starch, carrageenan, and a compound microbial inoculant. The compound microbial inoculant includes Bacillus subtilis and Lactobacillus rhamnosus.

### TECHNICAL FIELD

The present disclosure belongs to the technical field of functional food, in particular to a gel preparation, a method for making the same, and use thereof.

### BACKGROUND

Diabetes is a metabolic disease characterized primarily by hyperglycemia, and is classified as a chronic, lifelong metabolic disease. At the same time, risk factors associated with diabetes, such as hyperlipidemia and hypertension, are also common clinical features among diabetics. For example, 80% of patients with type II diabetes have severe dyslipidemia. Due to insufficient insulin, the lipase activity in vivo decreases, resulting in a significant increase in free fatty acids, triglycerides and cholesterol in the blood, which in turn leads to hyperlipidemia as a complication.

At present, there is no specific medicine capable of completely curing hyperglycemia and hyperlipidemia in the medical community. Patients with type II diabetes and hyperlipidemia can only reduce blood glucose and lipids by oral administration of western medicine or injection of insulin. However, western medicine usually has side effects, and long-term use will cause serious liver and kidney injury in patients. Insulin injections, while effective for lowering blood sugar, pose challenges such as inconvenience in administration, pain and discomfort, and potential for local skin induration with long-term use.

The whole inner surface of the stomach is covered with a layer of mucosal tissue, which can secrete mucus and form a mucus-mucosal barrier to protect the gastric tissue. Gastric mucosal injury is a common digestive disease worldwide. It is characterized by high recurrence rates, complex etiology and difficulty to completely cure. In severe cases, it can cause gastric ulcer or even gastric perforation, threatening human health. Factors such as stress, smoking, alcohol consumption, long-term use of non-steroidal anti-inflammatory drugs (NSAIDs), and Helicobacter pylori infection can all contribute to gastric mucosal injury, further leading to mucous edema, erosion or even bleeding and necrosis. Although the current ulcer protection drugs are effective, they have strong toxic and side effects.

With the improvement of living standards, increasing attention is paid to dietary therapy. Therefore, it is of great significance and broad prospects to develop a medicinal and edible product capable of maintaining blood glucose and lipid levels within a healthy range and assisting in protecting gastric mucosa.

### SUMMARY

In view of this, an objective of the embodiments of the present disclosure is to provide a gel preparation capable of maintaining blood glucose and lipid levels within a healthy range and assisting in protecting gastric mucosa, a preparation method and use thereof.

To achieve the above objective, the present disclosure provides the following technical solutions.

The present disclosure provides a gel preparation capable of maintaining blood glucose and lipid levels within a healthy range and assisting in protecting gastric mucosa. The gel preparation includes the following raw materials: 5-10 parts by weight of konjac, 9-15 parts by weight of a banana, 6-12 parts by weight of a watermelon, 8-15 parts by weight of dragon fruit peel, 8-14 parts by weight of fresh juice, 10-15 parts by weight of Caryophylli Flos, 8-12 parts by weight of Mori Folium Praeparata Cum Melle, 4-8 parts by weight of Sophorae Flos Carbonisatus, 16-20 parts by weight of starch, 2-4 parts by weight of carrageenan, and 0.5-1 part by weight of a compound microbial inoculant, where the compound microbial inoculant includes Bacillus subtilis and Lactobacillus rhamnosus, the fresh juice is composed of dragon fruit juice and lemon juice, and the dragon fruit juice and the lemon juice are used in a ratio of 1:1.

In some embodiments, the Bacillus subtilis and the Lactobacillus rhamnosus in the compound microbial inoculant have a weight ratio of 1:3-4.

In some embodiments, the Sophorae Flos Carbonisatus is processed by stir-frying Sophorae Flos on strong fire until ustulate, adding clean water, and stir-frying dry.

In some embodiments, the Mori Folium Praeparata Cum Melle is processed by mixing honey with Mori Folium, covered moistening, and stir-frying on mild fire until a dark yellow surface appears.

The present disclosure further provides a method for preparing the foregoing gel preparation, including the following steps: cooking the konjac, drying and pulverizing the cooked konjac to obtain konjac flour; crushing the banana and the watermelon, mixing the crushed banana and watermelon with the starch, performing steaming to obtain a mixture; mixing the Caryophylli Flos and the Mori Folium Praeparata Cum Melle with water, boiling, and collecting a supernatant to obtain an aqueous extract; mixing the konjac flour, the mixture, the aqueous extract, the Sophorae Flos Carbonisatus, the dragon fruit peel, the fresh juice, and the compound microbial inoculant for fermentation to obtain a fermentation product; and mixing the fermentation product with the carrageenan to obtain the gel preparation.

In some embodiments, the banana and the watermelon are unpeeled banana and watermelon.

In some embodiments, the steaming is conducted for 10-30 min.

In some embodiments, the water is 2-3 times the total weight of the Caryophylli Flos and the Mori Folium Praeparata Cum Melle.

In some embodiments, the fermentation is conducted at 18-23° C. for 24-48 h.

The present disclosure further provides use of the foregoing gel preparation or the foregoing preparation method in preparing a product for regulating blood glucose and/or blood lipids and controlling gastric mucosal injury.

Embodiments of the present disclosure has the following beneficial effects:

The present disclosure first provides a gel preparation capable of maintaining blood glucose and lipids at a healthy level and assisting in protecting gastric mucosa. Fruit raw materials (banana, watermelon, and dragon fruit peel), fresh juice, and cellulose material (konjac) are used in combination with medicinal and edible raw materials (Caryophylli Flos, Mori Folium Praeparata Cum Melle, and Sophorae Flos Carbonisatus) and starch; the foregoing raw materials are fermented with a compound microbial inoculant to efficiently integrate dietotherapeutic and medicinal effects; finally, the gel preparation obtained has a significant effect on regulating blood glucose and lipids, and protecting gastric mucosa from damage.

In addition, the gel preparation provided by the present disclosure is similar to jelly in appearance, with moderate sweet taste and no obvious medicinal taste. It has reddish color, and exhibits excellent flavor and taste, making it easily acceptable by consumers. It is non-toxic and demonstrates excellent dietotherapeutic effect.

### BRIEF DESCRIPTION OF THE DRAWINGS

FIG. 1 illustrates changes of serum cholesterol levels in different groups of mice;

FIG. 2 illustrates changes of serum triglycerides levels in different groups of mice;

FIG. 3 illustrates blood glucose levels of different groups of mice; and

FIG. 4 illustrates ulcer indexes of different groups of rats.

### DETAILED DESCRIPTION OF THE EMBODIMENTS

The present disclosure provides a gel preparation capable of maintaining blood glucose and lipids at a health level and assisting in protecting gastric mucosa. The gel preparation includes the following raw materials: 5-10 parts by weight of konjac, 9-15 parts by weight of a banana, 6-12 parts by weight of a watermelon, 8-15 parts by weight of dragon fruit peel, 8-14 parts by weight of fresh juice, 10-15 parts by weight of Caryophylli Flos, 8-12 parts by weight of Mori Folium Praeparata Cum Melle, 4-8 parts by weight of Sophorae Flos Carbonisatus, 16-20 parts by weight of starch, 2-4 parts by weight of carrageenan, and 0.5-1 part by weight of a compound microbial inoculant, where the compound microbial inoculant includes Bacillus subtilis and Lactobacillus rhamnosus, the fresh juice is composed of dragon fruit juice and lemon juice, and the dragon fruit juice and the lemon juice are used in a ratio of 1:1.

The present disclosure has no special limitation on specific sources of the foregoing raw materials. In the present disclosure, the dragon fruit peel may preferably be red-fleshed dragon fruit peel; the Mori Folium Praeparata Cum Melle Prominent may preferably be processed by mixing honey with Mori Folium, covered moistening, and stir-frying on mild fire until a dark yellow surface appears; the honey and the Mori Folium may preferably have a weight ratio of 1:5-7, and more preferably 1:6. In the present disclosure, the Sophorae Flos Carbonisatus may preferably be processed by stir-frying Sophorae Flos on strong fire until ustulate, adding clean water, and stir-frying dry. A quantity of the clean water may preferably be ⅕ to 1/7 of the quantity of the Sophorae Flos. In the present disclosure, the Bacillus subtilis and the Lactobacillus rhamnosus in the compound microbial inoculant may have a weight ratio of 1:3-4; the Bacillus subtilis may preferably have a viable count of 2×10¹⁰ CFU/g, and the Lactobacillus rhamnosus may preferably have a viable count of 4.5×10¹¹ CFU/g; the Lactobacillus rhamnosus may preferably be Lactobacillus rhamnosus HN001. The present disclosure has no special limitation on specific sources of the Bacillus subtilis and the Lactobacillus rhamnosus. In the present disclosure, the dragon fruit juice is obtained by peeling and juicing a dragon fruit in a juicer; the lemon juice is obtained by juicing an unpeeled lemon in the juicer. In the present disclosure, the gel preparation may preferably include the following raw materials: 7-9 parts by weight of the konjac, 11-13 parts by weight of the banana, 8-10 parts by weight of the watermelon, 10-12 parts by weight of the dragon fruit peel, 10-12 parts by weight of the fresh juice, 12-14 parts by weight of the Caryophylli Flos, 9-10 parts by weight of the Mori Folium Praeparata Cum Melle, 5-7 parts by weight of the Sophorae Flos Carbonisatus, 18-19 parts by weight of the starch, 3 parts by weight of the carrageenan, and 0.6-0.8 parts by weight of the compound microbial inoculant, where the compound microbial inoculant is composed of the Bacillus subtilis and the Lactobacillus rhamnosus HN001.

The present disclosure further provides a method for preparing the foregoing gel preparation, including the following steps: cooking the konjac, drying and pulverizing the cooked konjac to obtain konjac flour; crushing the banana and the watermelon, mixing the crushed banana and watermelon with the starch, and performing steaming to obtain a mixture; mixing the Caryophylli Flos and the Mori Folium Praeparata Cum Melle with water, boiling, and collecting a supernatant to obtain an aqueous extract; mixing the konjac flour, the mixture, the aqueous extract, the Sophorae Flos Carbonisatus, the dragon fruit peel, the fresh juice, and the compound microbial inoculant for fermentation to obtain a fermentation product; and mixing the fermentation product with the carrageenan to obtain the gel preparation.

In the present disclosure, the konjac is cooked before drying and pulverization. The drying may preferably be baking. The baking may preferably be conducted at 40-50° C. for 30-60 min and at 70-80° C. for 1-2 h in sequence. The present disclosure has no special limitation on a specific pulverization method. Pulverized konjac is preferably sieved through a 100-mesh sieve to obtain the konjac flour for later use.

In the present disclosure, the banana and the watermelon are unpeeled banana and watermelon. The method for crushing the unpeeled banana and the unpeeled watermelon may preferably be pulping. After the pulping, a mixed puree of the watermelon and the banana is steamed with the starch. The steaming may preferably be conducted for 10-30 min, and more preferably 15-20 min, to obtain a mixture for later use.

In the present disclosure, the Caryophylli Flos and the Mori Folium Praeparata Cum Melle are preferably pulverized and boiled in water. The present disclosure has no special limitation on a specific pulverization method. In some embodiments, pulverized Caryophylli Flos and Mori Folium Praeparata Cum Melle are needed to pass through a 40- to 60-mesh sieve. A quantity of the water may preferably be 2-3 times a total weight of the Caryophylli Flos and the Mori Folium Praeparata Cum Melle. The boiling may preferably be conducted for 20-30 min. After the boiling, centrifugation may preferably be conducted. The centrifugation may preferably be conducted at 800-1,000 r/min for 3-5 min, and more preferably 900 r/min for 4 min. A supernatant is collected to obtain an aqueous extract for later use.

In the present disclosure, the dragon fruit peel and the Sophorae Flos Carbonisatus are preferably crushed and mixed with the konjac flour, the mixture, the aqueous extract and the compound microbial inoculant for fermentation. In the present disclosure, the method for crushing the dragon fruit peel and the Sophorae Flos Carbonisatus may preferably be pulping. The puree is mixed with the konjac flour, the mixture, the aqueous extract and the compound microbial inoculant for fermentation. The fermentation may preferably be conducted at 18-23° C. for 24-48 h, and more preferably 19-22° C. for 30-40 h. In the preparation method described in the present disclosure, there is no sequential relationship in the preparation process of the konjac flour, the mixture and the aqueous extract, which can depend on the case.

The present disclosure further provides use of the foregoing gel preparation or the foregoing preparation method in preparing a product for regulating blood glucose and/or blood lipids and controlling gastric mucosal injury.

In the present disclosure, the type of the product may preferably be one selected from the group consisting of health products and medicaments.

The technical solutions provided by the present disclosure will be described in detail below with reference to examples, but the examples should not be construed as limiting the claimed scope of the present disclosure.

In the following examples, all experimental methods are conventional methods, unless otherwise specified.

All materials and reagents used in the following examples may be commercially available, unless otherwise specified.

### Example 1

A gel preparation capable of maintaining blood glucose and lipids at a healthy level and assisting in protecting gastric mucosa was made from the following raw materials: 5 g of konjac, 9 g of unpeeled banana, 6 g of unpeeled watermelon, 8 g of red-fleshed dragon fruit peel, 8 g of fresh juice (4 g of red-fleshed dragon fruit juice (obtained by peeling and juicing) and 4 g of lemon juice (obtained by juicing without peeling)), 10 g of Caryophylli Flos, 8 g of Mori Folium Praeparata Cum Melle (processed by mixing honey with Mori Folium (the honey and the Mori Folium had a weight ratio of 1:5), covered moistening, and stir-frying on mild fire until a dark yellow surface appears), 4 g of Sophorae Flos Carbonisatus (processed by stir-frying Sophorae Flos on strong fire until ustulate, adding clean water (the quantity of the clean water was ⅕ of the quantity of Sophorae Flos), and stir-frying dry), 16 g of starch, 2 g of carrageenan, and 0.5 g of compound microbial inoculant. The compound microbial inoculant was composed of 0.125 g of Bacillus subtilis (Shandong Yihao Biotechnology Co., Ltd., 2×10¹⁰ CFU/g) and 0.375 g of Lactobacillus rhamnosus HN001 (Xinyang Mufan Biotechnology Co., Ltd., MF-010611, 4.5×10¹¹ CFU/g).

The gel preparation was prepared as follows:

The konjac was washed, cooked, removed, dried at 40° C. for 30 min and at 70° C. for 2 h in sequence, and then crushed through a 100-mesh sieve to obtain konjac flour for later use.

The unpeeled banana and the unpeeled watermelon were washed and pulped in a pulper to obtain a mixed puree, and the mixed puree was mixed and steamed with starch for 20 min to obtain a mixture for later use.

The Caryophylli Flos and the Mori Folium Praeparata Cum Melle were pulverized and sieved through a 60-mesh sieve, and the powder was mixed with 36 g of water and boiled; afterwards, the boiling state was held for 20 min, followed by centrifugation at 800 r/min for 5 min; the supernatant was collected to obtain an aqueous extract for later use.

After the Sophorae Flos Carbonisatus and the red-fleshed dragon fruit peels were crushed, the puree was mixed with the konjac flour, the mixture, the aqueous extract, the fresh juice and the compound microbial inoculant, and fermented at 22° C. for 30 h to obtain a fermentation product; and

the fermentation product was mixed with carrageenan to obtain the gel preparation, and the gel preparation was filled; after filling, the gel preparation was sterilized in a 90° C. water bath for 10 min to obtain a finished gel preparation.

### Example 2

A gel preparation capable of maintaining blood glucose and lipids at a health level and assisting in protecting gastric mucosa was made from the following raw materials: 10 g of konjac, 15 g of unpeeled banana, 12 g of unpeeled watermelon, 15 g of red-fleshed dragon fruit peel, 14 g of fresh juice (7 g of red-fleshed dragon fruit juice (obtained by peeling and juicing) and 7 g of lemon juice (obtained by juicing without peeling)), 15 g of Caryophylli Flos, 12 g of Mori Folium Praeparata Cum Melle (commercially available), 8 g of Sophorae Flos Carbonisatus (commercially available), 20 g of starch, 4 g of carrageenan, and 1 g of compound microbial inoculant. The compound microbial inoculant was composed of 0.2 g of Bacillus subtilis (Shandong Yihao Biotechnology Co., Ltd., 2×10¹⁰ CFU/g) and 0.8 g of Lactobacillus rhamnosus HN001 (Xinyang Mufan Biotechnology Co., Ltd., MF-010611, 4.5×10¹¹ CFU/g).

The gel preparation was prepared as follows:
The konjac was washed, cooked, removed, dried at 50° C. for 50 min and at 80° C. for 1 h in sequence, and then crushed through a 100-mesh sieve to obtain konjac flour for later use;
the unpeeled banana and the unpeeled watermelon were washed and pulped in a pulper to obtain a mixed puree, and the mixed puree was mixed and steamed with starch for 30 min to obtain a mixture for later use;
the Caryophylli Flos and the Mori Folium Praeparata Cum Melle were pulverized and sieved through a 40-mesh sieve, and the powder was mixed with 81 g of water and boiled; after boiling, the boiling state was held for 30 min, followed by centrifugation at 1,000 r/min for 4 min; the supernatant was collected to obtain an aqueous extract for later use;
after the Sophorae Flos Carbonisatus and the red-fleshed dragon fruit peels were crushed, the puree was mixed with the konjac flour, the mixture, the aqueous extract, the fresh juice and the compound microbial inoculant, and fermented at 18° C. for 48 h to obtain a fermentation product; and
the fermentation product was mixed with carrageenan to obtain the gel preparation, and the gel preparation was filled; after filling, the gel preparation was sterilized in a 90° C. water bath for 10 min to obtain a finished gel preparation.

### Example 3

A gel preparation capable of maintaining blood glucose and lipids at a health level and assisting in protecting gastric mucosa was made from the following raw materials: 8 g of konjac, 12 g of unpeeled banana, 8 g of unpeeled watermelon, 12 g of dragon fruit peel, 12 g of fresh juice (6 g of red-fleshed dragon fruit juice (obtained by peeling and juicing) and 6 g of lemon juice (obtained by juicing without peeling)), 12 g of Caryophylli Flos, 10 g of Mori Folium Praeparata Cum Melle (commercially available), 6 g of Sophorae Flos Carbonisatus (commercially available), 18 g of starch, 3 g of carrageenan, and 0.8 g of compound microbial inoculant. The compound microbial inoculant was composed of 0.2 g of Bacillus subtilis (Shandong Yihao Biotechnology Co., Ltd., 2×10¹⁰ CFU/g) and 0.6 g of Lactobacillus rhamnosus HN001 (Xinyang Mufan Biotechnology Co., Ltd., MF-010611, 4.5×10¹¹ CFU/g).

The gel preparation was prepared as follows:
The konjac was washed, cooked, removed, dried at 45° C. for 40 min and at 80° C. for 1.5 h in sequence, and then crushed through a 100-mesh sieve to obtain konjac flour for later use;
the unpeeled banana and the unpeeled watermelon were washed and pulped in a pulper to obtain a mixed puree, and the mixed puree was mixed and steamed with starch for 15 min to obtain a mixture for later use;
the Caryophylli Flos and the Mori Folium Praeparata Cum Melle were pulverized and sieved through a 60-mesh sieve, and the powder was mixed with 44 g of water and boiled; after boiling, the boiling state was held for 25 min, followed by centrifugation at 900 r/min for 5 min; the supernatant was collected to obtain an aqueous extract for later use;
after the Sophorae Flos Carbonisatus and the red-fleshed dragon fruit peels were crushed, the puree was mixed with the konjac flour, the mixture, the aqueous extract, the fresh juice and the compound microbial inoculant, and fermented at 22° C. for 35 h to obtain a fermentation product; and
the fermentation product was mixed with carrageenan to obtain the gel preparation, and the gel preparation was filled; after filling, the gel preparation was sterilized in a 90° C. water bath for 10 min to obtain a finished gel preparation.

### Comparative Example 1

The difference from Example 3 was that no compound microbial inoculant was added and the fermentation step of the compound microbial inoculant was not included. The rest were the same as Example 3.

### Comparative Example 2

The difference from Example 3 was that Bacillus subtilis was not added. The rest were the same as Example 3.

### Comparative Example 3

The difference from Example 3 was that unpeeled bananas and unpeeled watermelons were not added. The rest were the same as Example 3.

### Comparative Example 4

The difference from Example 3 was that Caryophylli Flos and Mori Folium Praeparata Cum Melle were not subjected to a water extraction step, but the Caryophylli Flos and Mori Folium Praeparata Cum Melle powders were directly used. The rest were the same as Example 3.

### Comparative Example 5

The difference from Example 3 was that Sophorae Flos Carbonisatus was replaced with Sophorae Flos. The rest were the same as Example 3.

### Comparative Example 6

The difference from Example 3 was that Caryophylli Flos, Mori Folium Praeparata Cum Melle, and Sophorae Flos Carbonisatus were not added. The rest were the same as Example 3.

### Example 4

The gel preparations obtained from Examples 1 to 3 were subjected to chronic toxicity testing in mice

Eighty healthy C57BL/6 mice weighing 20-22 g, half male and half female, were acclimatized for 24 h and randomly divided into 4 groups: Example 1 group, Example 2 group, Example 3 group, and control group. For Examples 1 to 3 groups, gel preparations obtained from Examples 1 to 3 were administered at a dose of 2,000 mg/kg/day by gastric gavage. The control mice were administered the corresponding volume of distilled water. After gastric gavage, the mice were fed with conventional feed, and the gel preparations or distilled water was administered by gastric gavage every day. The experiment lasted for 6 months, no mice died, and no toxicity was found in the mice of Examples 1 to 3 groups. Therefore, it is demonstrated that the gel preparation provided by the present disclosure has no toxic and side effects and is safe and reliable in clinical applications.

After the testing, the mice were weighed, the orbital blood was drawn, and the serum glycolipid metabolism indexes were measured. After the mice were sacrificed, the heart, liver, kidneys, lungs, brain, spleen and gastrointestinal tract were dissected for histopathologic examination. The long-term toxicity test in mice showed that there were no toxic and side effects on the growth, body weight, brain, liver and kidney functions of mice after 6-month gastric gavage of the gel preparation provided by the present disclosure. No damage was found in the tissues, organs and circulatory system of mice. The Examples 1 to 3 groups were significantly different from the control group. This indicates that the gel preparation provided by the present disclosure is non-toxic and suitable for long-term consumption.

### Example 5

### Lipid-Lowering Test in Mice

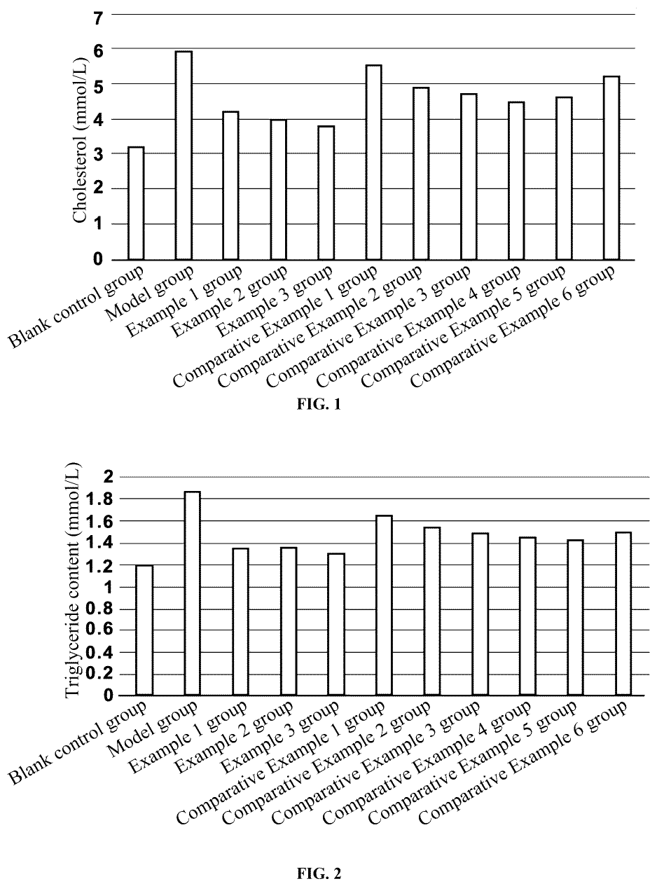

Two hundred and twenty healthy C57BL/6 mice weighing 22-24 g, half male and half female, were acclimatized for 24 h and randomly and equally divided into 11 groups: Examples 1 to 3 groups, Comparative Examples 1 to 6 groups, blank control (NC) group, and model group. Each group included 20 mice. The model group was fed a high-fat diet+distilled water (100 mg/kg/day) by gavage. The Examples 1 to 3 groups were fed a high-fat diet+gel preparation obtained from Examples 1 to 3 (100 mg/kg/day) by gavage. The Comparative Examples 1 to 6 groups were fed a high-fat diet+preparations obtained from Comparative Examples 1 to 6 (100 mg/kg/day) by gavage. The blank control group was administered the same volume of distilled water as that in Example 1 group by gavage. All mice were fed continuously for 10 weeks. The living conditions of the mice in each group were observed every week. After 10 weeks of feeding, mouse serum triglycerides and cholesterol were measured by orbital blood collection. The experimental results are shown in FIGS. 1 and 2.

It can be seen from FIGS. 1 and 2 that the daily administration of the gel preparation prepared in the present disclosure could effectively reduce the serum triglyceride and cholesterol levels of the mice. Although the serum triglycerides and cholesterol levels of the mice were lowered in the comparative example groups with a lack of types of raw materials and modified preparation methods, the lipid-lowering effect was obviously not as good as the gel preparation prepared by the present disclosure.

### Example 6

### Hypoglycemic Test in Mice

Modeling of streptozotocin (STZ)-induced diabetic mouse model: A total of 200 6-week-old C57 mice, half male and half female, were acclimatized for one week. Before the experiment, the mice were fasted for 12 h, and the STZ solution (pH 5.2 citric acid buffer) was injected intraperitoneally at a dose of 50 mg/kg for 5 consecutive days. The fasting blood glucose was tested at 1 and 2 weeks after injection, respectively. When the blood glucose value exceeded 11.0 mmol/L, the model was successfully established.

The model mice were randomly divided into 10 groups of 20 mice: model group, Examples 1 to 3 groups, and Comparative Examples 1 to 6 groups. Examples 1 to 3 groups and Comparative Examples 1 to 6 groups were administered the gel preparations prepared in the corresponding groups once a day by gavage at a dose of 100 mg/kg/day, and the model group was administered distilled water at a dose of 100 mg/kg/day every day. In addition, 20 6-week-old C57 mice, half male and half female, were selected and administered distilled water by gavage at a dose of 100 mg/kg/day as a blank control group. The administration to each group continued for 4 weeks, and then the gastric gavage was terminated.

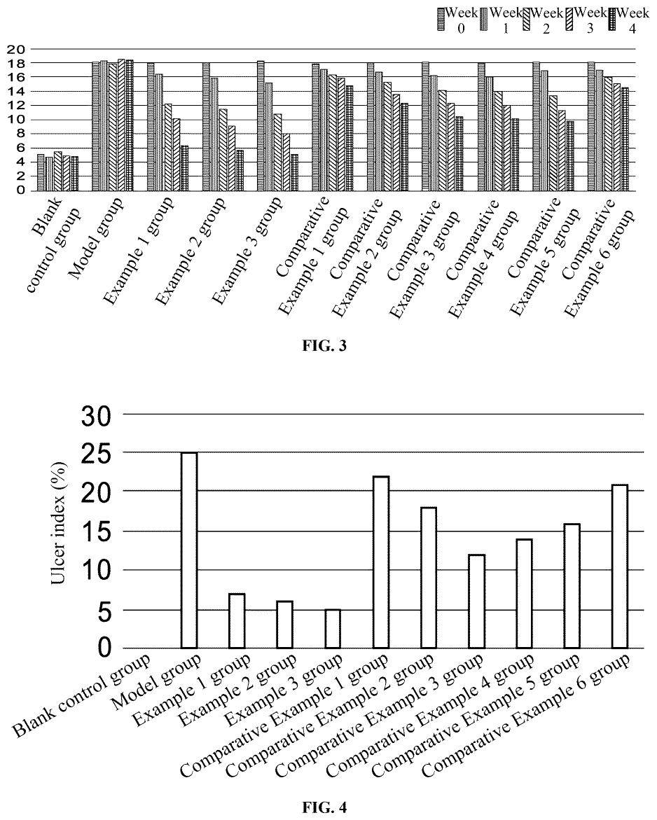

The mice were fasted for 8 h. The fasting began at 7:00 AM, and fasting blood glucose was tested from the tail vein at 3:00 PM by blood glucose meter (Sinocare GA-3 Blood Glucose Meter), serving as the blood glucose level at week 0 of administration. Afterwards, fasting blood glucose was measured at 1, 2, 3, and 4 weeks of administration, respectively. Fasting and testing times followed the same methods as those at week 0. The results are shown in FIG. 3. It can be seen that the gel preparation provided by the present disclosure can significantly reduce the fasting blood glucose of the STZ-induced diabetic mouse model, which is obviously better than that in Comparative Examples 1 to 6 groups.

### Example 7

The clinical observation of the treatment of type 2 diabetes with hyperlipidemia was conducted using gel preparations prepared in Example 3 and Comparative Examples 1 to 6.

### 1. Materials and Methods:

General information: Subjects were all recruited from the outpatient and inpatient departments of the Department of Endocrinology, Chengdu Integrated TCM and Western Medicine Hospital, Sichuan Province, and were grouped by statisticians using a simple random assignment method. The study was reviewed and approved by the Medical Clinical Trial Ethics Committee of Xinjin District Hospital of Traditional Chinese Medicine, Chengdu, and all subjects signed informed consent forms.

Treatment: A total of 180 eligible patients with type 2 diabetes were randomly divided into control group, simvastatin treatment group, Example 3 group, and Comparative Examples 1 to 6 groups, with 20 subjects in each group. Example 3 group took the gel preparation prepared from Example 3; Comparative Examples 1 to 6 groups took gel preparations (10 g, t.i.d., for 30 consecutive days) prepared from the corresponding comparative examples; the treatment group was treated with Simvastatin Dispersible Tablets (10 mg, q.d.), while insulin was added for treatment, and insulin glargine (initial dose 0.2 U/(kg-day), p.c.) was injected subcutaneously until the blood glucose levels of the patients returned to normal; the control group had access to normal diet and water in three meals a day.

### 2. Case Selection Criteria

Diagnostic criteria: The diagnostic criteria of type 2 diabetes were formulated with reference to the 2017 Standards of Medical Care in Diabetes (ADA). Traditional Chinese medicine (TCM) syndromes were formulated with reference to the Guidelines for Clinical Research on New Drugs (TCM) in the Treatment of Dispersion-thirst Disease (Diabetes), the Traditional Chinese Medicine Guideline for the Prevention and Treatment of Diabetes by the China Association of Chinese Medicine, and the diagnosis and treatment program for dispersion-thirst disease (type 2 diabetes) by the Key Specialist Cooperation Group of the National Administration of Traditional Chinese Medicine.

### Inclusion and Exclusion Criteria:

Inclusion criteria included:
(1) those who met the diagnostic criteria for type 2 diabetes: diabetic symptoms, with random plasma glucose (RPG) ≥11.1 mmol/L, fasting plasma glucose (FPG) ≥7.0 mmol/L, or 2-h postprandial plasma glucose (2 h-PG) during oral glucose tolerance test (OGTT) ≥11.1 mmol/L;
(2) patients with hyperlipemia: serum total cholesterol (TC) ≥5.18 mmol/L, low-density lipoprotein cholesterol (LDL-C) ≥3.37 mmol/L, or triglyceride (TG) ≥1.70 mmol/L, with or without high-density lipoprotein cholesterol (HDL-C)≤1.04 mmol/L;
(3) those who did not take other antihyperlipidemic drugs within 2 weeks before enrollment, with a need for a 2-week washout period in the presence of those who took other antihyperlipidemic drugs;
(4) patients who were on insulin or drugs for controlling a steady blood glucose level;
(5) those aged between 18 and 60;
(6) patients who had no desire of fertility over the last six months; and
(7) volunteers who signed informed consent forms.

Exclusion criteria included:
(1) patients with ketoacidosis and hyperosmotic nonketotic coma;
(2) those who were on other TCM decoctions and Chinese patent medicine for the treatment of diabetes concurrently;
(3) patients with other chronic diseases, hepatorenal diseases or liver and kidney dysfunctions;
(4) pregnant and lactating women; and
(5) those who participated in clinical trials of other drugs 3 months before the trial.

All patients must meet four of the above inclusion criteria without any of the five exclusion criteria before they were enrolled in this study.

### Observation Indices:

(1) Safety evaluation: Heart rate and rhythm; complete blood count (CBC), urinalysis, and stool routine test; liver and kidney panels; local and systemic adverse reactions, which were tested twice before and after treatment.
(2) Outcome measures and evaluation methods: Fasting blood glucose (FBG) and 2-h postprandial plasma glucose (2 h-PG) were primary outcome measures. The normal FBG value was 3.9-6.1 mmol/L; the normal 2 h-PG was less than 7.8 mmol/L.

As formulated by the Guidelines for Clinical Research of Cardiovascular Drugs of the Ministry of Health, marked response is defined as that lipid levels achieve any of the following: TC decreasing by ≥20%, LDL-C decreasing by ≥40%, or HDL-C increasing by ≥0.26 mmol/L; moderate response is defined as: TC decreasing by 10-20%, LDL-C decreasing by 20-40%, or HDL-C increasing by (0.104-0.25) mmol/L; and no response is defined as those who do not meet effective criteria.
(3) Response evaluation and time: The TCM symptoms, signs, FBG, 2 h-PG, TC, TG, LDL and HDL levels of patients were observed and recorded in detail before and after treatment.

Statistical methods: The clinical data were calculated by SPSS19.0 software, and the data were analyzed by statistical software SPSS16.0. The measurement data were expressed as (x±s), and the t test was used for comparison between groups. P<0.05 indicated that the difference was statistically significant.

### Results:

Safety evaluation: There was no significant change in heart rate and rhythm in the subjects. There was no abnormality in CBC, urinalysis, stool routine test, liver and kidney panels. No systemic adverse reactions occurred during the medication.

Both FBG and 2 h-PG were lower than those in the control group (P<0.05), as shown in Table 1.

After treatment, the overall response rate (marked response rate+moderate response rate) of each group of subjects was higher than that of the control group (P<0.05), as shown in Table 2.

TABLE 1
Results of blood glucose control of each group

| | | Example | Comparative | Comparative | Comparative | Comparative | Comparative | Comparative | Sim- | |
|---|---|---|---|---|---|---|---|---|---|---|
| | | 3 | Example 1 | Example 2 | Example 3 | Example 4 | Example 5 | Example 6 | vastatin | Control |
| FBG | Before | 14.1 ± 1.8 | 13.8 ± 1.9 | 14.3 ± 2.1 | 13.5 ± 1.7 | 14.5 ± 1.1 | 13.9 ± 2.8 | 14.1 ± 1.3 | 12.8 ± 1.6 | 13.2 ± 1.5 |
| | After | 5.8 ± 2.1 | 11.1 ± 1.6 | 9.8 ± 1.1 | 8.6 ± 1.4 | 7.6 ± 1.8 | 6.9 ± 1.6 | 10.5 ± 1.3 | 4.9 ± 1.2 | 13.9 ± 2.1 |
| 2h-PG | Before | 18.21 ± 1.3 | 18.35 ± 1.6 | 18.14 ± 1.4 | 18.2 ± 1.5 | 18.1 ± 1.6 | 18.15 ± 1.9 | 18.26 ± 2.1 | 18.24 ± 1.9 | 18.59 ± 1.7 |
| | After | 8.3 ± 1.9 | 16.22 ± 1.8 | 14.32 ± 1.6 | 13.22 ± 2.1 | 12.11 ± 1.5 | 11.76 ± 1.2 | 16.86 ± 1.4 | 7.8 ± 2.5 | 20.1 ± 1.4 |

TABLE 2
Comparison results of response rate of each group

| | | Marked | Moderate | No | Overall |
|---|---|---|---|---|---|
| Group | n | response | response | response | response |
| Example 3 group | 20 | 14 | 3 | 3 | 17 |
| Comparative | 20 | 3 | 1 | 16 | 4 |
| Example 1 | | | | | |
| Comparative | 20 | 6 | 5 | 9 | 11 |
| Example 2 | | | | | |
| Comparative | 20 | 7 | 6 | 7 | 13 |
| Example 3 | | | | | |
| Comparative | 20 | 8 | 5 | 7 | 13 |
| Example 4 | | | | | |
| Comparative | 20 | 9 | 5 | 6 | 14 |
| Example 5 | | | | | |
| Comparative | 20 | 4 | 4 | 12 | 8 |
| Example 6 | | | | | |
| Simvastatin | 20 | 16 | 3 | 1 | 19 |
| Control | 20 | 0 | 0 | 20 | 0 |

### Example 8

### Evaluation of Inhibitory Effects of Gel Preparations of Different Groups on Ethanol-Induced Gastric Injury in Sprague-Dawley (SD) Rats

Male SD rats were acclimatized for 24 h and randomly and equally divided into 11 groups: Examples 1 to 3 groups, Comparative Examples 1 to 6 groups, blank control group, and model group. Each group contained 10 mice. The Examples 1 to 3 groups and the Comparative Examples 1 to 6 groups were administered 100 mg/kg/day gel preparations prepared from Examples 1 to 3 and Comparative Examples 1 to 6, respectively. The blank control group and the model group were administered the same dose of normal saline by gavage.

The rats were deprived of feed and water. After 12 h, rats in each group were separately administered samples by gavage according the above grouping. After the samples were administered by gavage for 1 h, 4 mL/kg-bw 80% ethanol was given by gavage to model immediately. The blank control group was administered the same dose of normal saline by gavage. Blood was drawn from abdominal aorta after ether anesthesia. Rat gastric tissues were sampled, observed and photographed under a dissecting microscope. The area of gastric ulcer and the total area of glandular stomach were measured by Olympus Image pro plus, and the ulcer index was calculated: Ulcer index=(Total area of gastric ulcer/Total area of the corresponding glandular stomach)×100%. The experimental data were expressed as mean values, and SPSS software was used for data analysis to determine the presence of statistical significance between the groups.

The results are shown in FIG. 4. The ulcer index is significantly lower in the rats administered with the gel preparation of the present disclosure by gavage in advance than in the model group (P<0.05).

The above are merely preferred embodiments of the present disclosure. It should be noted that several improvements and modifications may further be made by a person of ordinary skill in the art without departing from the principle of the present disclosure, and such improvements and modifications should also be deemed as falling within the protection scope of the present disclosure.

What is claimed is:

1. A gel preparation capable of maintaining blood glucose and lipids within a healthy range and assisting in protecting gastric mucosa, comprising the following raw materials: 5-10 parts by weight of konjac, 9-15 parts by weight of a banana, 6-12 parts by weight of a watermelon, 8-15 parts by weight of dragon fruit peel, 8-14 parts by weight of fresh juice, 10-15 parts by weight of Caryophylli Flos, 8-12 parts by weight of Mori Folium Praeparata Cum Melle, 4-8 parts by weight of Sophorae Flos Carbonisatus, 16-20 parts by weight of starch, 2-4 parts by weight of carrageenan, and 0.5-1 part by weight of a compound microbial inoculant, wherein the compound microbial inoculant comprises Bacillus subtilis and Lactobacillus rhamnosus, the fresh juice comprises dragon fruit juice and lemon juice, and the dragon fruit juice and the lemon juice are used in a ratio of 1:1.

2. The gel preparation according to claim 1, wherein the Bacillus subtilis and the Lactobacillus rhamnosus in the compound microbial inoculant have a weight ratio of 1:3-4.

3. The gel preparation according to claim 1, wherein the Sophorae Flos Carbonisatus is processed by stir-frying Sophorae Flos on strong fire until ustulate, adding clean water, and stir-frying dry.

4. The gel preparation according to claim 1, wherein the Mori Folium Praeparata Cum Melle is processed by mixing honey with Mori Folium, covered moistening, and stir-frying on mild fire until a dark yellow surface appears.

5. A method for preparing the gel preparation according to claim 1, comprising the following steps: cooking the konjac, drying and pulverizing the cooked konjac to obtain konjac flour; crushing the banana and the watermelon, mixing the crushed banana and watermelon with the starch and conducting steaming to obtain a mixture; mixing the Caryophylli Flos and the Mori Folium Praeparata Cum Melle with water, boiling, and collecting a supernatant to obtain an aqueous extract; mixing the konjac flour, the mixture, the aqueous extract, the Sophorae Flos Carbonisatus, the dragon fruit peel, the fresh juice, and the compound microbial inoculant for fermentation to obtain a fermentation product; and mixing the fermentation product with the carrageenan to obtain the gel preparation.

6. The method according to claim 5, wherein the Bacillus subtilis and the Lactobacillus rhamnosus in the compound microbial inoculant have a weight ratio of 1:3-4.

7. The gel preparation according to claim 5, wherein the Sophorae Flos Carbonisatus is processed by stir-frying Sophorae Flos on strong fire until ustulate, adding clean water, and stir-frying dry.

8. The gel preparation according to claim 5, wherein the Mori Folium Praeparata Cum Melle is processed by mixing honey with Mori Folium, covered moistening, and stir-frying on mild fire until a dark yellow surface appears.

9. The method according to claim 5, wherein the banana and the watermelon are unpeeled banana and watermelon.

10. The method according to claim 5, wherein the steaming is conducted for 10-30 min.

11. The method according to claim 5, wherein a quantity of the water is 2-3 times a total weight of the Caryophylli Flos and the Mori Folium Praeparata Cum Melle.

12. The method according to claim 5, wherein the fermentation is conducted at 18-23° C. for 24-48 h.

13. A method for regulating blood glucose and/or blood lipids and controlling gastric mucosal injury, comprising administering to a subject in need there of a therapeutically effective amount of a product comprising the gel preparation according to claim 1.

14. The method according to claim 13, wherein the Bacillus subtilis and the Lactobacillus rhamnosus in the compound microbial inoculant have a weight ratio of 1:3-4.

15. The method according to claim 13, wherein the Sophorae Flos Carbonisatus is processed by stir-frying Sophorae Flos on strong fire until ustulate, adding clean water, and stir-frying dry.

16. The method according to claim 13, wherein the Mori Folium Praeparata Cum Melle is processed by mixing honey with Mori Folium, covered moistening, and stir-frying on mild fire until a dark yellow surface appears.
